(19) United States
(12) Patent Application Publication
(10) Pub. No.: US 2025/0214879 A1
(43) Pub. Date: Jul. 3, 2025

(54) WASTE TREATMENT SYSTEM AND METHOD OF CONVERTING WASTE INTO ENERGY

(71) Applicant: INDUSTRIAL TECHNOLOGY RESEARCH INSTITUTE, Hsinchu (TW)

(72) Inventors: Jia-Hung WANG, Hsinchu City (TW); Cheng-Chung HUANG, Taichung City (TW); Shing-Der CHEN, Chiayi City (TW); Laurensia IRMAYANI, Hsinchu City (TW); Li-Ching CHUNG, Zhubei City (TW); Rui-Cong SUN, Changhua City (TW); Wang-Kuan CHANG, Hsinchu City (TW)

(73) Assignee: INDUSTRIAL TECHNOLOGY RESEARCH INSTITUTE, Hsinchu (TW)

(21) Appl. No.: 18/740,642

(22) Filed: Jun. 12, 2024

(30) Foreign Application Priority Data
Dec. 28, 2023 (TW) 112151332

(51) Int. Cl. C02F 11/04; C02F 1/36; C02F 3/34; C02F 103/32; C02F 103/36; C10L 3/08

(52) U.S. Cl. CPC C02F 11/04; C02F 1/36; C02F 3/342; C02F 3/343; C02F 3/344; C02F 3/348; C10L 3/08; C02F 2103/322; C02F 2103/365; C02F 2203/004; C02F 2209/02; C02F 2209/06; C02F 2303/10

(57) ABSTRACT

A method of converting waste into energy is provided. The method includes the following steps: (a) providing a lipid-containing substrate to react with a biocatalyst to produce a surfactant molecular liquid; (b) pretreating an organic waste with the surfactant molecular liquid to produce a first organic liquid; (c) subjecting the first organic liquid to an ultrasonic treatment to produce a second organic liquid; and (d) subjecting the second organic liquid to an anaerobic biological treatment for conversion to methane. The biocatalyst includes at least one lipase. Moreover, the surfactant molecular liquid includes at least one of monoglyceride and diglyceride. A waste treatment system for converting waste into energy is also provided.

### CROSS REFERENCE TO RELATED APPLICATIONS

This application claims priority of Taiwan Patent Application No. 112151332, filed on Dec. 28, 2023, the entirety of which is incorporated by reference herein.

### TECHNICAL FIELD

The present invention relates to a waste treatment system and a method of converting waste into energy.

### BACKGROUND

The manufacturing industry contributes considerable output value to the global economy, but it also produces a large amount of waste. As the model of a circular economy becomes mainstream, how to convert waste into resources and reduce carbon emissions has gradually attracted attention from both industry and government.

Under the environmental protection demands of a sustainable environment, manufacturing industry sludge containing a large amount of organic waste is regarded as a recyclable resource. How to effectively process and utilize this waste sludge has become an important issue in the current manufacturing industry. Among current sludge treatment technologies, physical pretreatment methods based on ultrasonic waves have advantages in terms of reducing environmental pollution, but they still face challenges in terms of power consumption.

Based on the above, although existing waste sludge treatment technologies have generally met their intended use, they still do not fully meet the requirements in all respects. Therefore, the development of energy-saving waste sludge treatment methods that can shorten treatment time or improve treatment efficiency while maintaining ultrasonic treatment efficiency is still an issue of concern in related fields.

### SUMMARY

In accordance with some embodiments of the present disclosure, a method of converting waste into energy is provided. The method includes the following steps: (a) providing a lipid-containing substrate to react with a biocatalyst to produce a surfactant molecular liquid; (b) pretreating an organic waste with the surfactant molecular liquid to produce a first organic liquid; (c) subjecting the first organic liquid to an ultrasonic treatment to produce a second organic liquid; and (d) subjecting the second organic liquid to an anaerobic biological treatment for conversion to methane. The biocatalyst includes at least one lipase. The weight percentage (wt %) of the biocatalyst to the lipid-containing substrate is 0.005 to 0.02:1. In addition, the surfactant molecular liquid includes at least one of monoglyceride and diglyceride.

In accordance with some embodiments of the present disclosure, a waste treatment system is also provided. The waste treatment system includes a surfactant molecular liquid generator, an ultrasonic generator and an anaerobic bioreactor. The surfactant molecular liquid generator includes a biocatalyst for treating a lipid-containing substrate to produce a surfactant molecular liquid. The ultrasonic generator is connected to the surfactant molecular liquid generator. The ultrasonic generator is used to process an organic liquid produced by mixing an organic waste and the surfactant molecular liquid. The anaerobic bioreactor is connected to the ultrasonic generator. The anaerobic bioreactor is used to process the organic liquid to produce methane. The biocatalyst includes at least one lipase. The weight percentage (wt %) of the biocatalyst to the lipid-containing substrate is 0.005 to 0.02:1. In addition, the surfactant molecular liquid includes at least one of monoglyceride and diglyceride.

In order to make the features or advantages of the present disclosure clear and easy to understand, a detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 shows a schematic diagram of a waste treatment system in accordance with some embodiments of the present disclosure;

FIG. 2 shows a schematic diagram of the hydrolysis reaction of triglyceride catalyzed by lipase (EC 3.1.1.3);

FIG. 3 shows a step flow chart of a method of converting waste into energy in accordance with some embodiments of the present disclosure;

FIG. 4 shows the test results of lipid conversion rate of biocatalysts with lipases derived from different bacterial strains and different compositions in accordance with some embodiments of the present disclosure. BSL is a group of lipases derived from Bacillus subtilis. YLL is a group of lipases derived from Yarrowia lipolytica. ANL is a group of lipases derived from Aspergillus niger. BSL+YLL+ANL (1:1:1) is a group of lipases derived from Bacillus subtilis, Yarrowia lipolytica and Aspergillus niger, and the weight percentage of the lipases derived from Bacillus subtilis, Yarrowia lipolytica and Aspergillus niger used is 1:1:1. BSL+YLL+ANL (1:2:3) is a group of lipases derived from Bacillus subtilis, Yarrowia lipolytica and Aspergillus niger, and the weight percentage of the lipases derived from Bacillus subtilis, Yarrowia lipolytica and Aspergillus niger used is 1:2:3;

FIG. 5 and FIG. 6 show the analysis results of testing the effect of the addition of surfactant molecular liquid on ultrasonic treatment of organic waste in accordance with some embodiments of the present disclosure;

FIG. 7 shows the analysis results of the effect of the addition of surfactant molecular liquid on the anaerobic biological treatment (methanogenesis potential) of organic waste in accordance with some embodiments of the present disclosure.

### DETAILED DESCRIPTION

The waste treatment system and the method of converting waste into energy of the present disclosure are described in detail in the following description. It should be understood that in the following detailed description, for purposes of explanation, numerous specific details and embodiments are set forth in order to provide a thorough understanding of the present disclosure. The specific elements and configurations described in the following detailed description are set forth in order to clearly describe the present disclosure. It will be apparent that the exemplary embodiments set forth herein are used merely for the purpose of illustration and not the limitation of the present disclosure.

The embodiments of the present disclosure can be understood together with the drawings, and the drawings of the present disclosure are also regarded as part of the disclosure descriptions. It should be understood that the drawings of the present disclosure are not drawn to scale and, in fact, the dimensions of elements may be arbitrarily enlarged or reduced in order to clearly illustrate features of the present disclosure.

Unless defined otherwise, all technical and scientific terms used herein have the same meaning as commonly understood by one of ordinary skill in the art to which this disclosure belongs. It should be appreciated that, in each case, the term, which is defined in a commonly used dictionary, should be interpreted as having a meaning that conforms to the relative skills of the present disclosure and the background or the context of the present disclosure, and should not be interpreted in an idealized or overly formal manner unless so defined.

In view of the waste sludge reduction demand faced by the current industry and the market demand for related technical products and services, embodiments of the present disclosure provide a method of converting waste into energy. The method uses biocatalyst catalysis technology combined with ultrasonic sludge treatment technology and further combines resource-to-energy conversion units to generate biomass methane, thereby establishing a waste treatment system with high treatment efficiency and energy saving. In accordance with the embodiments of the present disclosure, the method of converting waste into energy and the waste treatment system can accelerate the conversion of organic waste into biomass methane for reuse. They can reduce the processing cost of organic waste, and increase the output of green electricity from biomass, while achieving the carbon reduction effect of waste reduction and the energy conversion of waste resources.

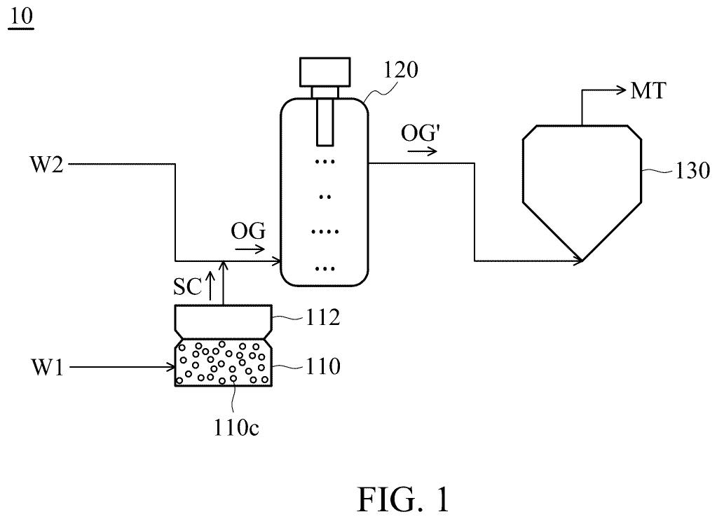

FIG. 1 shows a schematic diagram of a waste treatment system 10 in accordance with some embodiments of the present disclosure. It should be understood that, for clarity of explanation, some elements of the waste treatment system 10 are omitted in the drawings, and only some elements are schematically illustrated. In accordance with some embodiments, additional features may be added to the waste treatment system 10 described below.

Referring to FIG. 1, the waste treatment system 10 may include a surfactant molecular liquid generator 110, an ultrasonic generator 120 and an anaerobic bioreactor 130. The ultrasonic generator 120 may be connected to the surfactant molecular liquid generator 110, and the anaerobic bioreactor 130 may be connected to the ultrasonic generator 120. The ultrasonic generator 120 may be located downstream of the surfactant molecular liquid generator 110, and the anaerobic bioreactor 130 may be located downstream of the ultrasonic generator 120. In accordance with some embodiments, the aforementioned surfactant molecular liquid generator 110, ultrasonic generator 120 and anaerobic bioreactor 130 may be connected through pipelines.

The surfactant molecular liquid generator 110 may include a biocatalyst 110c, and the biocatalyst 110c may be used to treat a lipid-containing substrate W1 to produce a surfactant molecular liquid SC. In accordance with some embodiments, the lipid-containing substrate W1 may be provided by a substrate providing unit (not illustrated), and the substrate providing unit may be connected to the surfactant molecular liquid generator 110.

In accordance with some embodiments, the lipid-containing substrate W1 may include a medium-and-long chain triglyceride (MLCT) with a carbon number of C12-C20, for example, may be the triglyceride with a carbon number of C12, C14, C16, C18 or C20, but it is not limited thereto. In accordance with some embodiments, the lipid-containing substrate W1 may include a long-chain triglyceride with a carbon number of C16-C20. In accordance with some embodiments, the lipid-containing substrate W1 may include food-industry wastewater, manufacturing wastewater, edible oil, feed oil, recycled oil of the aforementioned oils, another suitable oil, or a combination thereof, but it is not limited thereto.

The biocatalyst 110c may include at least one lipase. Furthermore, the lipase may be immobilized on a carrier, and the base material of the carrier may include chitosan or another suitable carrier base material, but it is not limited thereto. In accordance with some embodiments, the lipase may include triglyceride lipase (EC 3.1.1.3), but it is not limited thereto. In accordance with some embodiments, the lipase may be derived from at least one of Aspergillus niger, Yarrowia lipolytica, and Bacillus subtilis. In accordance with some embodiments, the lipases may be derived from Aspergillus niger, Yarrowia lipolytica, and Bacillus subtilis, and the weight percentage of the lipases respectively derived from Aspergillus niger, Yarrowia lipolytica, and Bacillus subtilis may be 1 to 3:1 to 3:1 to 3. In particular, the lipases derived from multiple bacterial strains can provide better broad-spectrum effects and improve the efficiency of lipases in catalyzing lipid hydrolysis.

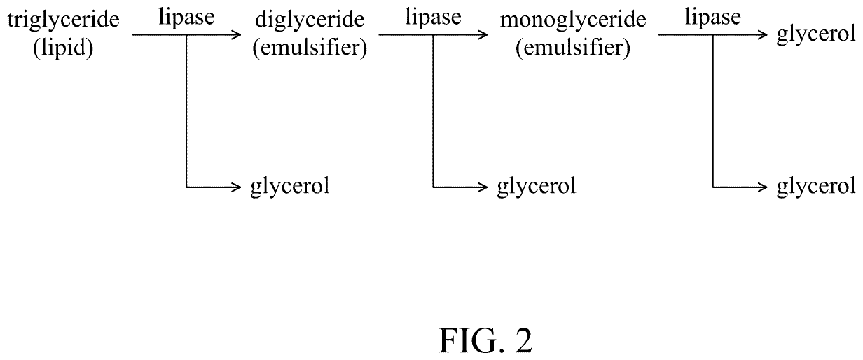

The surfactant molecular liquid SC produced by the lipid-containing substrate W1 through the action of lipase refers to a liquid with surfactant properties. The surfactant molecular liquid SC may include at least one of monoglyceride and diglyceride. For example, please refer to FIG. 2, which shows a schematic diagram of the hydrolysis reaction of triglyceride catalyzed by lipase (EC 3.1.1.3). Lipase acts on the ester bond of lipid molecules, which can hydrolyze triglyceride into diglyceride and fatty acid, and can subsequently hydrolyze diglyceride into monoglyceride and fatty acid, and can further hydrolyze monoglyceride into glycerol and fatty acid. It should be noted that the monoglyceride and diglyceride in the surfactant molecular liquid SC have emulsifier-like functions, and when subsequently acting on an organic waste W2, the solubility and homogeneity of the organic waste W2 can be increased. The surface tension of the mixture to be treated also can be reduced, and the time required for subsequent ultrasonic treatment can be shortened. Furthermore, free fatty acid molecules can serve as precursor nutrients for anaerobic biological conversion into methane, which can increase the production of biomass methane in the subsequent treatment stage of the anaerobic bioreactor 130.

Furthermore, the weight percentage (wt %) of the biocatalyst to the lipid-containing substrate W1 may be 0.005 to 0.02:1, for example, may be 0.006:1, 0.007:1, 0.008:1, 0.009:1, 0.01:1, 0.011:1, 0.012:1, 0.013:1, 0.014:1, 0.015:1, 0.016:1, 0.017:1, 0.018:1 or 0.019:1, but it is not limited thereto. In particular, if the ratio of the biocatalyst to the lipid-containing substrate W1 is too low (for example, less than 0.005:1), the time required for the lipase to act may be too long, thereby reducing the processing efficiency of the surfactant molecular liquid generator 110. On the contrary, if the ratio of the biocatalyst to the lipid-containing substrate W1 is too high (for example, higher than 0.02:1), the production cost may be greatly increased.

In accordance with some embodiments, the surfactant molecular liquid generator 110 may perform a reaction at a temperature of 25° C. to 45° C. and a pH of 6.5 to pH 7.5, so that the lipid-containing substrate W1 reacts with the biocatalyst 110c to produce surfactant molecular liquid SC. In accordance with some embodiments, the reaction temperature of the surfactant molecular liquid generator 110 may be from 25° C. to 40° C., or from 25° C. to 35° C., for example, may be 26° C., 27° C., 28° C., 29° C., 30° C., 31° C., 32° C., 33° C. or 34° C., but it is not limited thereto. In accordance with some embodiments, the reaction pH of the surfactant molecular liquid generator 110 may be pH 6.6, pH 6.7, pH 6.8, pH 6.9, pH 7, pH 7.1, pH 7.2, pH 7.3, or pH 7.4, but it is not limited thereto.

Furthermore, in accordance with some embodiments, the waste treatment system 10 may further include a surfactant molecular liquid storage tank 112, which may be connected to the surfactant molecular liquid generator 110 for temporarily storing the surfactant molecular liquid SC. The surfactant molecular liquid storage tank 112 may be further connected to the ultrasonic generator 120 to transport the surfactant molecular liquid SC to the ultrasonic generator 120.

In detail, before being transported to the ultrasonic generator 120, the surfactant molecular liquid SC may be mixed with the organic waste W2, and the surfactant molecular liquid SC may pretreat the organic waste W2 to produce a first organic liquid OG. The treated first organic liquid OG may be decomposed into a homogeneous organic liquid with smaller molecules, such as organic sludge.

In accordance with some embodiments, the organic waste W2 may be provided by a waste providing unit (not illustrated), and the waste providing unit may be connected to the ultrasonic generator 120. Specifically, the pipeline of the waste providing unit may be connected to the pipeline of the surfactant molecular liquid generator 110 and then connected together to the ultrasonic generator 120.

In accordance with some embodiments, the organic waste W2 may include manufacturing waste, petrochemical industry waste, agricultural waste, animal husbandry waste, food waste, another suitable organic waste or a combination thereof, but it is not limited thereto.

As described above, the surfactant molecular liquid SC, generated after the lipid-containing substrate W1 is processed by the surfactant molecular liquid generator 110, may include at least one of monoglyceride and diglyceride. Monoglyceride and diglyceride have emulsifier-like functions, which can increase the solubility and homogeneity of the organic waste W2, reduce the surface tension of the mixture to be treated, and shorten the time required for subsequent ultrasonic treatment.

In accordance with some embodiments, in the homogeneous first organic liquid OG, the volume percentage (v/v %) of the surfactant molecular liquid SC to the organic waste W2 may be 0.005 to 0.05:1, for example, may be 0.01:1, 0.015:1, 0.02:1, 0.025:1, 0.03:1, 0.035:1, 0.04:1 or 0.045:1, but it is not limited thereto. It should be noted that when the ratio of the surfactant molecular liquid SC to the organic waste W2 is within the aforementioned range, the treatment efficiency of the surfactant molecular liquid SC for the organic waste W2 can be effectively improved.

In accordance with some embodiments, the pretreatment of organic waste W2 by surfactant molecular liquid SC may be performed in a continuous reaction at a temperature from 20° C. to 60° C. and a pH from pH 5 to pH 8. In accordance with some embodiments, the reaction temperature of the aforementioned pretreatment may be from 30° C. to 50° C., or from 30° C. to 40° C., for example, may be 22° C., 24° C., 26° C., 27° C., 28° C., 29° C., 30° C., 31° C., 32° C., 33° C., 34° C., 35° C., 36° C., 37° C., 38° C., 39° C., 42° C., 45° C., 48° C., 52° C., 55° C. or 58° C., but it is not limited thereto. In accordance with some embodiments, the reaction pH of the aforementioned pretreatment may be from pH 6.5 to pH 7.5, for example, may be pH 6.6, pH 6.7, pH 6.8, pH 6.9, pH 7, pH 7.1, pH 7.2, pH 7.3 or pH 7.4, but it is not limited thereto.

Furthermore, the ultrasonic generator 120 may be used to process the first organic liquid OG produced by mixing the organic waste W2 and the surfactant molecular liquid SC to produce a second organic liquid OG′. The ultrasonic generator 120 can apply ultrasonic energy to the first organic liquid OG to hydrolyze organic matter and improve the efficiency of subsequent biological anaerobic treatment in the anaerobic bioreactor 130. Specifically, the impact force generated by the cavitation effect provided by ultrasonic waves can destroy the structure of the first organic liquid OG (for example, the cell wall of microorganisms in organic sludge), causing the concentration of dissolved organic matter in the first organic liquid OG to increase. This makes it easier for subsequent anaerobic microorganisms to digest, thereby shortening the time required for biological anaerobic treatment.

In accordance with some embodiments, the output power of the ultrasonic treatment may be from 300 watts to 1200 watts, for example, may be 400 watts, 500 watts, 600 watts, 700 watts, 800 watts, 900 watts, 1000 watts or 1100 watts, but it is not limited thereto. In accordance with some embodiments, the frequency of ultrasonic treatment may be from 20 kilohertz (kHz) to 100 kHz, for example, may be 30 kHz, 40 kHz, 50 kHz, 60 kHz, 70 kHz, 80 kHz or 90 kHz, but it is not limited thereto.

The anaerobic bioreactor 130 may be used to process the second organic liquid OG′ to produce methane MT, and the methane MT can be converted into electricity by subsequent biogas power generation facilities. Specifically, the anaerobic biological treatment can decompose and transform small molecular organic matter through the biochemical metabolism of microorganisms to produce biogas, such as methane. In accordance with some embodiments, the anaerobic bioreactor 130 may include hydrolytic bacteria, acid-forming bacteria, methanogens, other suitable bacterial species, or a combination thereof, but it is not limited thereto.

In accordance with some embodiments, the anaerobic biological treatment performed in the anaerobic bioreactor 130 may be performed at a temperature from 25° C. to 45° C. and a pH from pH 6.8 to pH 7.2. In accordance with some embodiments, the reaction temperature of the aforementioned anaerobic biological treatment may be from 30° C. to 40° C., for example, may be 26° C., 27° C., 28° C., 29° C., 30° C., 31° C., 32° C.° C., 33° C., 34° C., 35° C., 36° C., 37° C., 38° C., 39° C., 40° C., 41° C., 42° C., 43° C. or 44° C., but it is not limited thereto. In accordance with some embodiments, the reaction pH of the aforementioned anaerobic biological treatment may be pH 6.9, pH 7, or pH 7.1, but it is not limited thereto.

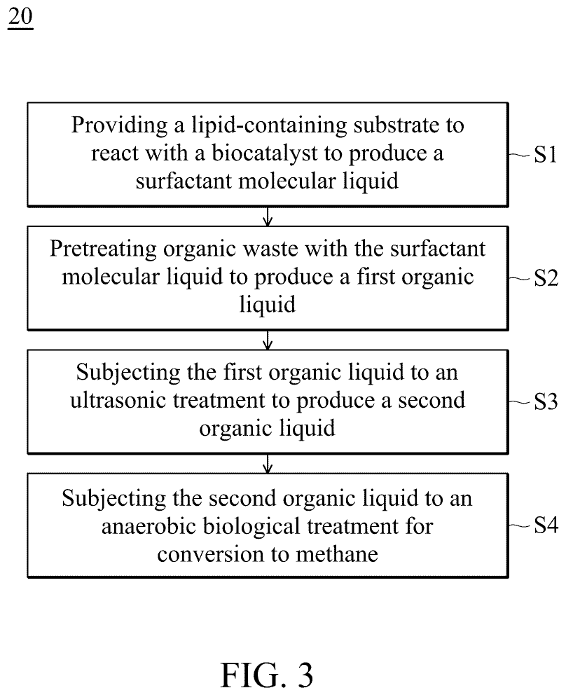

In addition, a method of converting waste into energy 20 is also provided in the present disclosure. FIG. 3 shows a step flow chart of the method of converting waste into energy 20 in accordance with some embodiments of the present disclosure. In accordance with some embodiments, the method of converting waste into energy 20 may include using the aforementioned waste treatment system 10 to process organic waste, but the present disclosure is not limited thereto. It should be understood that, in accordance with some embodiments, additional steps may be provided before, during and/or after the method of converting waste into energy 20 described below, or some steps may be replaced or omitted.

As shown in FIG. 3, the method of converting waste into energy 20 may include step S1: providing a lipid-containing substrate W1 to react with a biocatalyst 110c to produce a surfactant molecular liquid SC.

In accordance with some embodiments, the lipid-containing substrate W1 may include medium-and-long chain triglycerides with a carbon number of C12-C20, for example, the triglycerides with a carbon number of C12, C14, C16, C18 or C20, but it is not limited thereto. In accordance with some embodiments, the lipid-containing substrate W1 may include long-chain triglycerides with a carbon number of C16-C20. In accordance with some embodiments, the lipid-containing substrate W1 may include food-industry wastewater, manufacturing wastewater, edible oil, feed oil, recycled oil of the aforementioned oils, another suitable oil, or a combination thereof, but it is not limited thereto.

The biocatalyst 110c may include at least one lipase. Furthermore, the lipase may be immobilized on a carrier, and the base material of the carrier may include chitosan or another suitable carrier base material, but it is not limited thereto. In accordance with some embodiments, the lipase may include triglyceride lipase (EC 3.1.1.3), but it is not limited thereto. In accordance with some embodiments, the lipase may be derived from at least one of Aspergillus niger, Yarrowia lipolytica, and Bacillus subtilis. In accordance with some embodiments, the lipases may be derived from Aspergillus niger, Yarrowia lipolytica, and Bacillus subtilis, and the weight percentage of the lipases respectively derived from Aspergillus niger, Yarrowia lipolytica, and Bacillus subtilis may be 1 to 3:1 to 3:1 to 3, for example, may be 1:1:1, 1:1:2, 1:1.5:2, 1:1.8:2.5, 1:2:3, 1:2.5:3, 1:3:2, 1:3:1.5, 2:1:2, 2:1:2.5, 2:1:3, 2:2:1, 2:2.5:1.8, 2:3:1, 3:1:2, 3:1.8:1.5, 3:2:1, 3:2.5:1, 3:3:1 or 3:3:2, but it is not limited thereto. In particular, the lipases derived from multiple bacterial strains can provide better broad-spectrum effects and improve the efficiency of lipases in catalyzing lipid hydrolysis. Furthermore, the lipases derived from the above-mentioned specific bacterial strains have good pairing properties with substrates, and have good catalytic performance especially for medium-and-long chain triglycerides with a carbon number of C12-C20.

The surfactant molecular liquid SC produced by the lipid-containing substrate W1 through the action of lipase may include at least one of monoglyceride and diglyceride. The monoglyceride and diglyceride in surfactant molecular liquid SC have emulsifier-like functions, which can increase the solubility and homogeneity of the organic waste W2 when subsequently reacting with the organic waste W2, reduce the surface tension of the mixture to be treated, and shorten the time required for subsequent ultrasonic treatment. Furthermore, free fatty acid molecules can serve as precursor nutrients for anaerobic biological conversion into methane, which can increase biomass methane production.

Furthermore, the weight percentage (wt %) of the biocatalyst to the lipid-containing substrate W1 may be 0.005 to 0.02:1, for example, may be 0.006:1, 0.007:1, 0.008:1, 0.009:1, 0.01:1, 0.011:1, 0.012:1, 0.013:1, 0.014:1, 0.015:1, 0.016:1, 0.017:1, 0.018:1 or 0.019:1, but it is not limited thereto. In particular, if the ratio of the biocatalyst to the lipid-containing substrate W1 is too low (for example, less than 0.005:1), the time required for the lipase to act may be too long, thereby reducing the processing efficiency of the surfactant molecular liquid generator 110. On the contrary, if the ratio of the biocatalyst to the lipid-containing substrate W1 is too high (for example, higher than 0.02:1), the production cost may be greatly increased.

In accordance with some embodiments, step S1 may be reacted at a temperature from 25° C. to 45° C. and a pH from pH 6.5 to pH 7.5 for 1 hour to 9 hours. In accordance with some embodiments, the reaction temperature of step S1 may be from 25° C. to 40° C., or from 25° C. to 35° C., for example, may be 26° C., 27° C., 28° C., 29° C., 30° C., 31° C., 32° C., 33° C., 34° C., 35° C., 36° C., 37° C., 38° C., 39° C., 40° C., 41° C., 42° C., 43° C. or 44° C., but it is not limited thereto. In accordance with some embodiments, the reaction pH of step S1 may be pH 6.6, pH 6.7, pH 6.8, pH 6.9, pH 7, pH 7.1, pH 7.2, pH 7.3 or pH 7.4, but it is not limited thereto. In accordance with some embodiments, the reaction time of step S1 may be 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours or 8.5 hours, but it is not limited thereto.

In addition, in accordance with some embodiments, step S1 may further include adjusting the lipid content of the lipid-containing substrate W1 to 30 wt % to 50 wt %, for example, it may be adjusted to 35 wt %, 40 wt % or 45 wt %, but it is not limited thereto. It should be noted that if the lipid content in the lipid-containing substrate W1 is too high (for example, higher than 50 wt %), the lipase may not be able to function effectively, resulting in poor fat hydrolysis.

Furthermore, the method of converting waste into energy 20 may include step S2: pretreating an organic waste W2 with a surfactant molecular liquid SC to produce a first organic liquid OG.

In accordance with some embodiments, the organic waste W2 may include manufacturing waste, petrochemical industry waste, agricultural waste, animal husbandry waste, food waste, another suitable organic waste or a combination thereof, but it is not limited thereto.

As described above, the surfactant molecular liquid SC may include at least one of monoglyceride and diglyceride. Since monoglyceride and diglyceride have emulsifier-like functions, the solubility and homogeneity of the organic waste W2 can be increased, the surface tension of the mixture to be treated can be reduced, and the time required for subsequent ultrasonic treatment can be shortened.

In accordance with some embodiments, in step S2, the volume percentage (v/v %) of the surfactant molecular liquid SC to the organic waste W2 may be 0.005 to 0.05:1, for example, may be 0.01:1, 0.015:1, 0.02:1, 0.025:1, 0.03:1, 0.035:1, 0.04:1 or 0.045:1, but it is not limited thereto. It should be noted that when the ratio of the surfactant molecular liquid SC to the organic waste W2 is within the aforementioned range, the treatment efficiency of the surfactant molecular liquid SC for the organic waste W2 can be effectively improved.

In accordance with some embodiments, step S2 performs a continuous reaction at a temperature from 20° C. to 60° C. and a pH from pH 5 to pH 8. In accordance with some embodiments, the reaction temperature of step S2 may be from 30° C. to 50° C., or from 30° C. to 40° C., for example, may be 22° C., 24° C., 26° C., 27° C., 28° C., 29° C., 30° C., 31° C., 32° C., 33° C., 34° C., 35° C., 36° C., 37° C., 38° C., 39° C., 42° C., 45° C., 48° C., 52° C., 55° C. or 58° C., but it is not limited thereto. In accordance with some embodiments, the reaction pH of step S2 may be from pH 6.5 to pH 7.5, for example, may be pH 6.6, pH 6.7, pH 6.8, pH 6.9, pH 7, pH 7.1, pH 7.2, pH 7.3 or pH 7.4, but it is not limited thereto.

Furthermore, the method of converting waste into energy 20 may include step S3: subjecting the first organic liquid OG to an ultrasonic treatment to produce a second organic liquid OG′.

In accordance with some embodiments, the output power of the ultrasonic treatment may be from 300 watts to 1200 watts, for example, may be 400 watts, 500 watts, 600 watts, 700 watts, 800 watts, 900 watts, 1000 watts or 1100 watts, but it is not limited thereto. In accordance with some embodiments, the frequency of ultrasonic treatment may be from 20 kHz to 100 kHz, for example, may be 30 kHz, 40 kHz, 50 kHz, 60 kHz, 70 kHz, 80 kHz or 90 kHz, but it is not limited thereto.

Moreover, the method of converting waste into energy 20 may include step S4: subjecting the second organic liquid OG′ to an anaerobic biological treatment for conversion to methane MT.

In accordance with some embodiments, step S4 may include using hydrolytic bacteria, acid-forming bacteria, methanogens, other suitable bacterial species or a combination thereof to perform biological anaerobic treatment, but it is not limited thereto. In accordance with some embodiments, step S4 may be performed at a temperature from 25° C. to 45° C. and a pH from pH 6.8 to pH 7.2. In accordance with some embodiments, the reaction temperature of step S4 may be from 30° C. to 40° C., for example, may be 26° C., 27° C., 28° C., 29° C., 30° C., 31° C., 32° C., 33° C., 34° C., 35° C., 36° C., 37° C., 38° C., 39° C., 40° C., 41° C., 42° C., 43° C. or 44° C., but it is not limited thereto. In accordance with some embodiments, the reaction pH of step S4 may be pH 6.9, pH 7, or pH 7.1, but it is not limited thereto.

In order to make the above-mentioned and other purposes, features and advantages of the present disclosure more thorough and easy to understand, a number of examples, comparative examples and testing examples are given below, and are described in detail as follows, but they are not intended to limit the scope of the present disclosure.

### Example 1—Preparation of Biocatalysts

1% acetic acid was used to dissolve chitosan to prepare a 2% chitosan solution. A syringe was used to add the chitosan solution dropwise to 10% sodium hydroxide solution, and the chitosan solution was condensed in the solution to form chitosan gel microspheres. The chitosan gel microspheres were placed at room temperature to harden for 60 minutes, and then were rinsed repeatedly with deionized water until neutral. Next, the aforementioned chitosan gel microspheres and 500 U/g lipase solution (EC3.1.1.3, from Aspergillus niger, Yarrowia lipolytica and Bacillus subtilis, and the composition ratio of the three is 1:1:1) relative to the concentration of carriers were cross-linked and immobilized with 0.25% glutaraldehyde solution. After 180 minutes, the chitosan gel microspheres containing immobilized lipase were taken out and washed. Compared with the free enzyme, the obtained immobilized enzyme has an enzyme activity preservation rate of 72.6%. After 10 batches of lipid hydrolysis reaction tests, the preservation rate of the immobilized enzyme can still reach 88.2% compared with the enzyme activity at the beginning of the lipid hydrolysis reaction tests. It can be seen that the biocatalysts prepared in the embodiments of the present disclosure have good enzyme activity and stability and are suitable for waste treatment systems.

### Example 2—Preparation of Surfactant Molecular Liquid

The food factory wastewater containing medium and long chain triglycerides was used as a lipid-containing substrate, and the lipid content thereof was adjusted to a range from 30 wt % to 50 wt %. The aforementioned lipid-containing substrate was used as a substrate material for preparing surfactant molecular liquid. Next, the lipid-containing substrate was reacted with the biocatalysts prepared in Example 1. The weight percentage (wt %) of the biocatalyst to the lipid-containing substrate was 0.005 to 0.02:1. The conversion reaction was carried out in batches for 1 to 3 hours at a temperature from 25° C. to 45° C. and a pH from pH 6.5 to pH 7.5, catalytically producing the surfactant molecular liquid containing monoglycerides, diglycerides and free fatty acid molecules.

### Example 3—Lipid Conversion Rate Test of the Biocatalysts Having Lipases of Different Bacterial Strain Sources and Compositions

The food factory wastewater obtained from the same source as Example 2 was used as a lipid-containing substrate, and the lipid content thereof was diluted to 50 wt %. The aforementioned lipid-containing substrate was used as the substrate material for this reaction test. Furthermore, 5 groups of biocatalysts with bacterial strain sources and compositions as shown in Table 1 below were established.

TABLE 1

| Group | Bacterial strain source of lipases | Composition |
|---|---|---|
| 1 | Bacillus subtilis | 100% |
| 2 | Yarrowia lipolytica | 100% |
| 3 | Aspergillus niger | 100% |
| 4 | Bacillus subtilis:Yarrowia | 1:1:1 |
| | lipolytica:Aspergillus niger | |
| 5 | Bacillus subtilis:Yarrowia | 1:2:3 |
| | lipolytica:Aspergillus niger | |

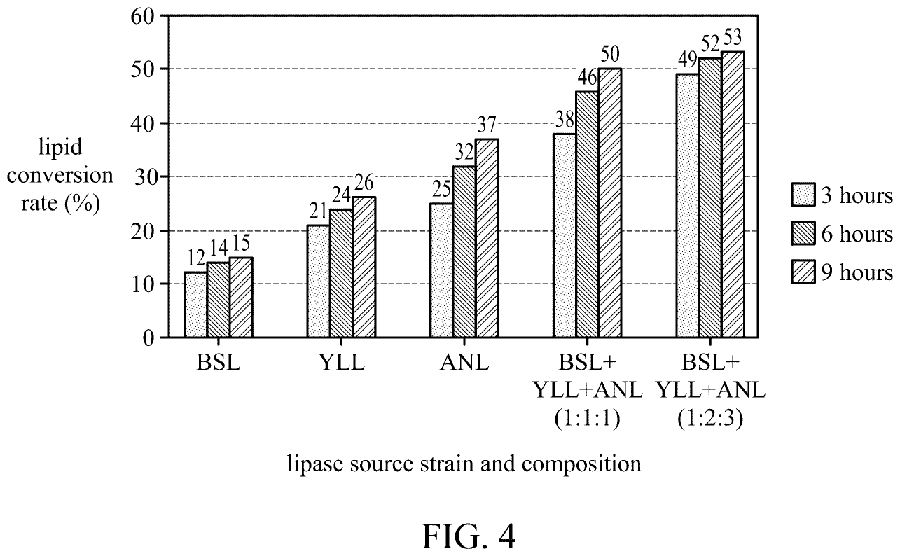

The test was divided into five groups, 100 g of the aforementioned lipid-containing substrate was taken as the substrate for reaction, and 2 g of the aforementioned five different biocatalysts of different compositions were added respectively. These mixtures were stirred evenly for reaction under the conditions of temperature 30° C. and pH 7.0. After the reaction time reached 3 hours, 6 hours and 9 hours, 1 ml of each of the reaction mixture was taken for lipid decomposition rate analysis to obtain the lipid conversion rate data of the above five different compositions of biocatalysts. The results are shown in FIG. 4. “BSL” is group 1 of lipases derived from Bacillus subtilis. “YLL” is group 2 of lipases derived from Yarrowia lipolytica. “ANL” is group 3 of lipases derived from Aspergillus niger. “BSL+YLL+ANL (1:1:1)” is group 4 of lipases derived from Bacillus subtilis, Yarrowia lipolytica and Aspergillus niger (the weight percentage of the three is 1:1:1). “BSL+YLL+ANL (1:2:3)” is group 5 of lipases derived from Bacillus subtilis, Yarrowia lipolytica and Aspergillus niger (the weight percentage of the three is 1:2:3).

As shown in FIG. 4, the lipid conversion rates of BSL derived from a single bacterial strain after reaction time of 3 hours, 6 hours and 9 hours were 12%, 14% and 15% respectively. The lipid conversion rates of YLL derived from a single bacterial strain after reaction time of 3 hours, 6 hours and 9 hours were 21%, 24% and 26% respectively. The lipid conversion rates of ANL derived from a single bacterial strain after reaction time of 3 hours, 6 hours and 9 hours were 25%, 32% and 37% respectively. The lipid conversion rates of BSL+YLL+ANL (1:1:1) derived from multiple bacterial strains after reaction time of 3 hours, 6 hours and 9 hours were 38%, 46% and 50% respectively. The lipid conversion rates of BSL+YLL+ANL (1:2:3) derived from multiple bacterial strains after reaction time of 3 hours, 6 hours and 9 hours were 49%, 52% and 53% respectively.

According to the foregoing results, it can be seen that biocatalysts with lipases derived from a single bacterial strain and multiple bacterial strains can catalyze the conversion of lipids. In addition, biocatalysts of lipases derived from various bacterial strains have better catalytic conversion efficiency, and the one containing a high proportion of Aspergillus niger (BSL+YLL+ANL (1:2:3)) has even better effects.

### Example 4—Test on the Lipid Conversion Rate of Biocatalyst Using Different Types of Oils as Substrate Materials

Two types of oils with different carbon chain lengths, olive oil (a long-chain triglyceride, LCT) and coconut oil (a medium-chain triglyceride, MCT), were used as substrates for this reaction test, and the biocatalysts of BSL+YLL+ANL (1:1:1) derived from multiple bacterial strains as described in Example 3 were added to prepare surfactant molecular liquids. After the reaction, the conversion rates of these two oils with different chain lengths were analyzed.

The test was divided into two groups, and 100 g of the water sample containing 50 wt % olive oil and 100 g of the water sample containing 50 wt % coconut oil were taken as the substrates of this reaction test. 2 g of BSL+YLL+ANL (1:1:1) biocatalysts derived from multiple bacterial strains as described in Example 3 were added to the water samples respectively, and were stirred evenly for 3 hours under the conditions of temperature 30° C. and pH 7.0. After 3 hours of reaction, 1 ml of the reaction mixture was taken out for lipid decomposition rate analysis to obtain the lipid conversion rate data when the above two different oil types were used as substrate materials.

The results showed that the lipid conversion rate of the olive oil (LCT) water sample was about 51%, and the lipid conversion rate of the coconut oil (MCT) water sample was about 23%. From the above results, it can be seen that both long-carbon chain lipids and medium-carbon chain lipids can be used as substrate materials for biocatalysts derived from multiple bacterial strains.

### Example 5—Organic Waste Decomposition Test

The waste solid sludge taken from the petrochemical industry was used as organic waste. The organic waste was mixed with the surfactant molecular liquid prepared in the aforementioned Example 2 to form a homogeneous organic liquid. The organic liquid was subjected to ultrasonic treatment, and then the chemical oxygen demand (COD) (i.e. concentration of dissolved organic matter, unit: mg/L) of the reaction hydrolyzate after ultrasonic treatment was measured to determine the effect of ultrasonic pretreatment.

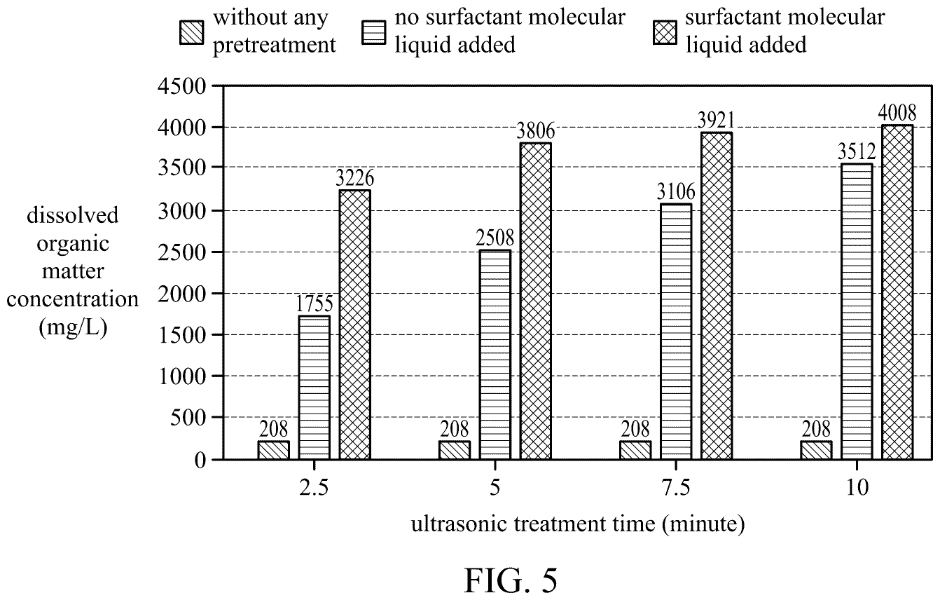

After 250 ml of organic sludge was respectively mixed with 250 ml of pure water and 250 ml of surfactant molecular liquid, the concentration of suspended solids (SS) in the organic sludge was measured to be 10256 mg/L, and the concentration of volatile suspended solids (VSS) was 7423 mg/L. Then, ultrasonic wave with a frequency of 20 kHz and a power of 500 watts were used for 2.5 minutes, 5 minutes, 7.5 minutes and 10 minutes in sequence. The results are shown in FIG. 5.

FIG. 5 shows the dissolved organic matter concentration analysis results of experimental samples of “without any pretreatment” (organic sludge with pure water added, but no ultrasonic treatment was performed), “no surfactant molecular liquid added” (organic sludge with pure water added, and subjected to ultrasonic treatment), and “surfactant molecular liquid added” (organic sludge with surfactant molecular liquid added, and subjected to ultrasonic treatment). The samples of the “no surfactant molecular liquid added” and “surfactant molecular liquid added” groups were treated with ultrasonic for 2.5 minutes, 5 minutes, 7.5 minutes, and 10 minutes respectively.

As shown in FIG. 5, the dissolved organic matter concentration of the sample “without any pretreatment” was maintained at 208 mg/L. The dissolved organic matter concentrations of the “no surfactant molecular liquid added” samples after being treated by ultrasonic for 2.5 minutes, 5 minutes, 7.5 minutes and 10 minutes were 1755 mg/L, 2508 mg/L, 3106 mg/L and 3512 mg/L respectively. The dissolved organic matter concentrations of the “surfactant molecular liquid added” samples after being treated by ultrasonic for 2.5 minutes, 5 minutes, 7.5 minutes and 10 minutes were 3226 mg/L, 3806 mg/L, 3921 mg/L and 4008 mg/L respectively.

Compared with the dissolved organic matter concentration of 3512 mg/L of the sample “no surfactant molecular liquid added” after ultrasonic treatment for 10 minutes, the dissolved organic matter concentration of the sample “surfactant molecular liquid added” after ultrasonic treatment for 10 minutes increased to 4008 mg/L, and the decomposition effect of organic waste increased by about 14%.

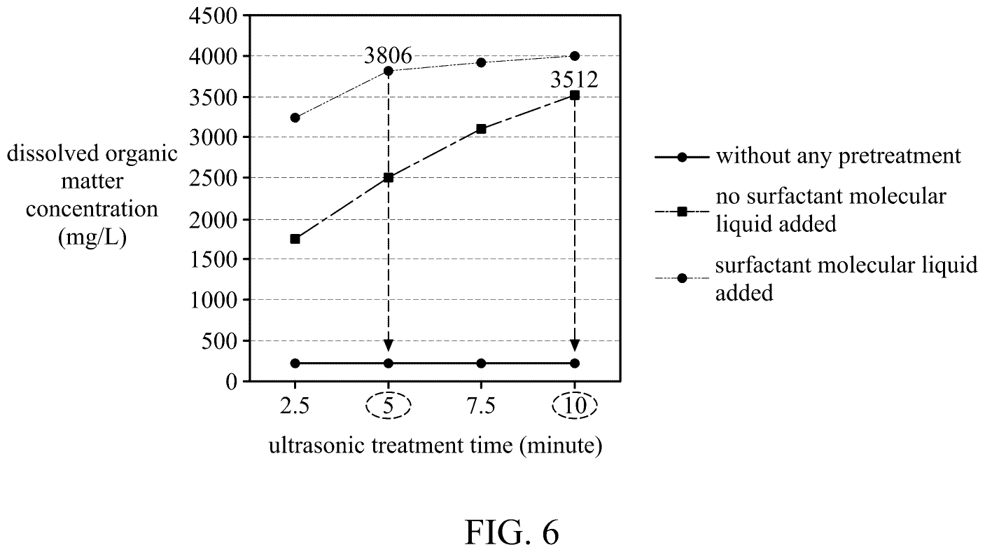

Next, please refer to FIG. 6, which converts the experimental results of FIG. 5 into a line graph. As shown in FIG. 6, the dissolved organic matter concentration of the sample “surfactant molecular liquid added” after ultrasonic treatment for 5 minutes was 3806 mg/L (energy consumption 42W), which has exceeded the concentration 3512 mg/L of the sample “no surfactant molecular liquid added” after ultrasonic treatment for 10 minutes (energy consumption 83W).

From the foregoing results, it can be seen that compared with the ultrasonic sludge treatment method that does not use biocatalysts (no surfactant molecular liquid added), the treatment method using biocatalyst catalysis combined with ultrasonic sludge treatment (surfactant molecular liquid added) requires only half the energy consumption (reduced from 83 W to 42W) and can increase the dissolved organic matter concentration of the material to be treated to 3512 mg/L, effectively improving treatment efficiency by more than 50%.

### Example 6—Methanogenesis Potential Test

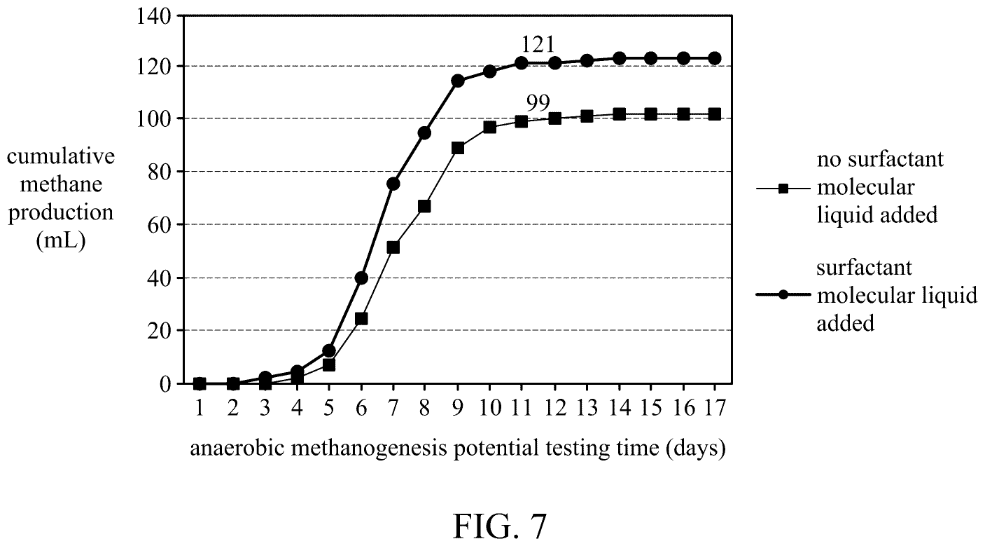

The test was divided into two groups. Three identical 600 ml reaction bottles were prepared for each group. The reaction bottles of two groups were respectively added with thoroughly mixed 350 ml of organic hydrolyzed sludge matrix obtained in Example 5 (group (1) “no surfactant molecular liquid added” and group (2) “surfactant molecular liquid added”, both groups were subjected to ultrasonic treatment for 5 minutes) and 150 ml of planting sludge (the planting sludge was taken from the tank of the anaerobic biological treatment unit of the wastewater treatment plant of the food factory, which contains general anaerobic bacteria such as hydrolytic bacteria, acidifying bacteria and methanogen). The reaction mixtures were shaken and stirred at a temperature of 35° C. and pH 7.0 for more than 21 days to conduct batch anaerobic digestion experiments. A gas collection hole was disposed at the top of the cap of the reaction bottle. The gas generated by the decomposition of organic hydrolyzed sludge was collected every day using the method of gas collection by water displacement, and the cumulative gas output of each test sample was recorded. The results are shown in FIG. 7.

As shown in FIG. 7, when the anaerobic biological treatment was carried out to the 11th day, the reaction was substantially completed. The cumulative gas production of the organic waste hydrolyzed sludge of the group “no surfactant molecular liquid added” was 99 ml. The cumulative gas production of the organic waste hydrolyzed sludge of the group “surfactant molecular liquid added” was 121 ml. It is estimated that compared with the sample of “no surfactant molecular liquid added”, the methane output of the sample “surfactant molecular liquid added” increased by about 22%, which effectively improved the efficiency of converting waste into energy.

To summarize the above, in accordance with the embodiments of the present disclosure, the provided method of converting waste into energy uses biocatalyst catalysis technology combined with ultrasonic sludge treatment technology and further combines resource-to-energy conversion units to generate biomass methane, thereby establishing a waste treatment system with high treatment efficiency and energy saving. In accordance with the embodiments of the present disclosure, the method of converting waste into energy and the waste treatment system can accelerate the conversion of organic waste into biomass methane for reuse. They can reduce the processing cost of organic waste, and increase the output of green electricity from biomass, while realizing the carbon reduction effect of waste reduction and the energy conversion of waste resources.

Although some embodiments of the present disclosure and their advantages have been described as above, it should be understood that various changes, substitutions and alterations can be made herein without departing from the spirit and scope of the disclosure as defined by the appended claims. In addition, each claim constitutes an individual embodiment, and the claimed scope of the present disclosure also includes the combinations of the claims and embodiments. The scope of protection of the present disclosure is subject to the definition of the scope of the appended claims.

What is claimed is:

1. A method of converting waste into energy, comprising the following steps:
(a) providing a lipid-containing substrate to react with a biocatalyst to produce a surfactant molecular liquid;
(b) pretreating an organic waste with the surfactant molecular liquid to produce a first organic liquid;
(c) subjecting the first organic liquid to an ultrasonic treatment to produce a second organic liquid; and
(d) subjecting the second organic liquid to an anaerobic biological treatment for conversion to methane,
wherein the biocatalyst comprises at least one lipase, the weight percentage (wt %) of the biocatalyst to the lipid-containing substrate is 0.005 to 0.02:1, and the surfactant molecular liquid comprises at least one of monoglyceride and diglyceride.

2. The method of converting waste into energy as claimed in claim 1, wherein the lipid-containing substrate comprises food-industry wastewater, manufacturing wastewater, edible oil, feed oil, recycled oil of the aforementioned oils, or a combination thereof.

3. The method of converting waste into energy as claimed in claim 1, wherein the lipid-containing substrate comprises a medium-and-long chain triglyceride (MLCT) with a carbon number of C12-C20.

4. The method of converting waste into energy as claimed in claim 1, wherein step (a) further comprises adjusting the lipid content of the lipid-containing substrate to 30 wt % to 50 wt %.

5. The method of converting waste into energy as claimed in claim 1, wherein step (a) is performed at a temperature from 25° C. to 45° C. and a pH from pH 6.5 to pH 7.5 for 1 hour to 9 hours.

6. The method of converting waste into energy as claimed in claim 1, wherein the lipase comprises triglyceride lipase (EC 3.1.1.3).

7. The method of converting waste into energy as claimed in claim 6, wherein the lipase is derived from at least one of Aspergillus niger, Yarrowia lipolytica, and Bacillus subtilis.

8. The method of converting waste into energy as claimed in claim 7, wherein the lipase is from Aspergillus niger, Yarrowia lipolytica, and Bacillus subtilis, and the weight percentage of the lipases respectively derived from Aspergillus niger, Yarrowia lipolytica, and Bacillus subtilis is 1 to 3:1 to 3:1 to 3.

9. The method of converting waste into energy as claimed in claim 1, wherein the lipase is immobilized on a carrier, and the base material of the carrier comprises chitosan.

10. The method of converting waste into energy as claimed in claim 1, wherein the organic waste comprises manufacturing waste, petrochemical industry waste, agricultural waste, animal husbandry waste, food waste or a combination thereof.

11. The method of converting waste into energy as claimed in claim 1, wherein in step (b), the volume percentage of the surfactant molecular liquid to the organic waste is 0.005 to 0.05:1.

12. The method of converting waste into energy as claimed in claim 1, wherein step (b) is a continuous reaction performed at a temperature from 20° C. to 60° C. and a pH from pH 5 to pH 8.

13. The method of converting waste into energy as claimed in claim 1, wherein in step (c), the output power of the ultrasonic treatment is from 300 watts to 1200 watts, and the frequency is from 20 kHz to 100 kHz.

14. A waste treatment system, comprising:
a surfactant molecular liquid generator, comprising a biocatalyst for treating a lipid-containing substrate to produce a surfactant molecular liquid;
an ultrasonic generator, connected to the surfactant molecular liquid generator, to process an organic liquid produced by mixing an organic waste and the surfactant molecular liquid; and
an anaerobic bioreactor, connected to the ultrasonic generator, to process the organic liquid to produce methane,
wherein the biocatalyst comprises at least one lipase, the weight percentage (wt %) of the biocatalyst to the lipid-containing substrate is 0.005 to 0.02:1, and the surfactant molecular liquid comprises at least one of monoglyceride and diglyceride.

15. The waste treatment system as claimed in claim 14, wherein the lipid-containing substrate comprises a triglyceride with a carbon number of C12-C20.

16. The waste treatment system as claimed in claim 14, wherein the surfactant molecular liquid generator performs reactions at a temperature from 25° C. to 45° C. and a pH from pH 6.5 to pH 7.5.

17. The waste treatment system as claimed in claim 14, wherein the lipase comprises triglyceride lipase (EC 3.1.1.3).

18. The waste treatment system as claimed in claim 17, wherein the lipase is derived from at least one of Aspergillus niger, Yarrowia lipolytica, and Bacillus subtilis.

19. The waste treatment system as claimed in claim 18, wherein the lipase is derived from Aspergillus niger, Yarrowia lipolytica, and Bacillus subtilis, and the weight percentage of the lipases respectively derived from Aspergillus niger, Yarrowia lipolytica, and Bacillus subtilis is 1 to 3:1 to 3:1 to 3.

20. The waste treatment system as claimed in claim 14, wherein the surfactant molecular liquid pretreats the organic waste to produce the organic liquid, and the pretreatment is performed in a continuous reaction at a temperature from 20° C. to 60° C. and a pH from pH 5 to pH 8.

21. The waste treatment system as claimed in claim 14, wherein the output power of the ultrasonic treatment is from 300 watts to 1200 watts, and the frequency is from 20 kHz to 100 kHz.
